(19) United States
(12) Patent Application Publication
(10) Pub. No.: US 2013/0338014 A1
(43) Pub. Date: Dec. 19, 2013

(54) Calibration Standards For Digital Histocytometry

(71) Applicant: Vala Sciences, Inc., (US)

(72) Inventors: Patrick M. McDonough, San Diego, CA (US); Jeffrey H. Price, San Diego, CA (US); Claire R. Weston, Poway, CA (US)

(73) Assignee: Vala Sciences, Inc., San Diego, CA (US)

(21) Appl. No.: 13/857,954

(22) Filed: Apr. 5, 2013

Related U.S. Application Data
(60) Provisional application No. 61/620,897, filed on Apr. 5, 2012.

(51) Int. Cl. G01N 33/569

(52) U.S. Cl. CPC G01N 33/569
USPC 506/4; 506/36; 506/30; 506/14

(57) ABSTRACT

An analytical reference for digital histochemistry is constituted of an on-slide standard used to calibrate biomarker expression in biopsy material and tissue sections. The standard consists of one or more calibration standards, which are derived from cell lines that express biomarkers, and are arranged into a calibration array. Expression of the desired biomarkers is validated by Western blotting, flow cytometry, or other methods. The calibration array is applied to a microscopy slide in such a manner that test samples from biopsies or tissue sections, or other cellular material, can also be mounted on the same slide, adjacent to the calibration array. Data obtained from the calibration array can be used to quantify expression of biomarkers in the biopsy material, tissue sections, tissue microarrays, and cellular material, via digital histochemistry.

### PRIORITY

This application claims priority to U.S. provisional application for patent 61/620,897, filed 04/05/2012.

### STATEMENT OF GOVERNMENT INTEREST

An invention described herein was made in part with government support under Contract No. HHSN261201000088C awarded by the National Institutes of Health. The United States government has certain rights in this invention.

### BACKGROUND

The field is immunohistochemical marking of biological material for analytical purposes. More particularly, the field concerns immunohistochemical standardization.

Immunohistochemistry denotes the use of immunohistochemical (IHC) dyes to label elements of biological tissue so as to visualize cellular structures in situ. NC staining can utilize chromogenic or fluorescent labels. Fluorescence-based staining is frequently referred to as immunofluoresence (IF). Digital histocytometry involves the use of an automated microscopy platform equipped with analytical tools to image cellular structures of an immunohistochemically-stained tissue sample mounted on a carrier such as a microscope slide and to measure features in the imaged cellular structures. The analytical tools are typically embodied in algorithms that perform image acquisition and measurement procedures, and that visualize, quantify, and analyze cellular structures, populations, and activities.

In many types of cancer, the expression level of certain proteins (biomarkers) within the tumor often determine the best course of treatment from among several options. To determine the expression of the biomarker(s) (there may be only a single biomarker of interest, or multiple biomarkers of interest), a tumor or portion of a tumor is collected by a biopsy procedure; the biopsy material is then processed so that it is appropriate for mounting on a histology slide (typically this involves cutting the biopsy material into sections and mounting the sections on to a slide). The histology slide is then visualized for expression of the biomarker by IHC or IF, through use of primary antibodies that specifically bind to a biomarker of interest, and secondary antibodies that are used to visualize the primary antibody molecules bound to the primary antibodies in the sample (secondary antibodies are coupled to enzymes that produce a colorimetric stain that is visible with bright field microscopy, or with fluorescent molecules that can be visualized and quantified via fluorescence (also called “dark-field) microscopy).

Biomarker expression is also important for determining efficacy and toxicity of candidate drugs in the pharmaceutical industry, and in basic science research. These studies are often carried out using tissue samples from humans and animals.

Most commonly, the expression level of biomarkers with biopsy material or tissue sections is visually “scored” by pathologists or other technicians. A disadvantage of this approach is that it is “semi-quantitative” at best, and subjective, as individual pathologists are known to disagree even when scoring the same tissue section (Renshaw, A. A. and E. W. Gould, Comparison of disagreement and amendment rates by tissue type and diagnosis: identifying cases for directed blinded review. Am J Clin Pathol, 2006. 126(5): p. 736-9).

An example that illustrates the use of biomarkers in the clinical diagnosis of cancer is breast cancer. Breast cancer tumors commonly feature the overexpression of estrogen receptor (ER) and the interaction of estrogen with the ER receptor is a stimulus for growth of the tumor. Accordingly, growth of such tumors can slowed or inhibited with medications that inhibit the interaction of estrogen with ER (e.g., ER antagonists) or reduce the expression of estrogen (inhibitors of enzymes that produce estrogen within the breast). It is therefore useful to ascertain the ER status of the tumor. Furthermore, the responsiveness of the tumor can depend not only upon whether or not ER is present, but the degree of expression of ER by the tumor. For example, the responses to tamoxifen (an agent that interferes with binding of estrogen to ER), and to letrozole (an agent that interferes with local production of estrogen within the breast) depend strongly on the degree of expression of ER by pathologists using a semiquantitative, scoring system, based upon visual inspection of tumor biopsies labeled for ER (Ellis, M. J., et al., Letrozole is more effective neoadjuvant endocrine therapy than tamoxifen for ErbB-1-and/or ErbB-2-positive, estrogen receptor-positive primary breast cancer: evidence from a phase III randomized trial. J Clin Oncol, 2001. 19(18): p. 3808-16).

It is likely that new quantitative methods, based upon using on-slide calibration samples, will improve the reproducibility of determining cellular ER expression.

Other biomarkers critical to determining optimal treatment strategies for breast cancer include the progesterone receptor (PR), and HER2/neu. PR is often expressed along with ER in breast tumors, and thus provides an additional biomarker for assisting in diagnosing which tumors will respond to anti-estrogen therapeutics. HER2/neu is a protein whose expression designates tumors that are susceptible to therapeutics that target HER2/neu (e. g., trastuzumab). Since tumors that express HER2/neu (also known as ErbB2) are relatively malignant, and since their growth can be slowed or inhibited by tratuzumab (also known as Herceptin®), it is important to determine if breast tumors express HER2/neu and the degree of HER2/neu expression.

Tissue sections from well-characterized samples are sometimes used on “reference slides” for calibration of the immunohistochemistry system. The problem with this strategy is that protein expression within a tissue is heterogeneous. This means that serial sections cut sequentially from the same reference tissue will have different patterns of biomarker expression. This, and the limited material available from a reference tissue, limits the use of serial sections from the same tissue as a source of calibration slides.

A calibration standard is a reference that enables the analytical tools of digital histocytometry to quantify biomarkers in tissue, such as, for example, clinical formalin fixed paraffin embedded tissue (FFPET) sections. There is an acute need for IHC calibration standards due to the increasing number of key biomarkers, the fact that often only a few cells need be positive to alter the course of treatment, and the inherently subjective and laborious nature of current diagnostic pathology practice.

More particularly, there is a need for on-slide calibration standards that allow the analytical tools of digital histocytometry to quickly, reliably, and reproducibly quantify biomarkers in biopsies and tissue sections.

### SUMMARY

In some aspects, the problems relating to manufacturability, cost, and consistency of calibration standards are solved by culturing large numbers of cells in separate culture flasks, and then mixing cells from different flasks together to obtain a single mixture. The amounts and distributions of a given biomarker (also called an analyte) are measured using an aliquot of each separate cell mixture via a standard technique such as flow cytometry, Western blotting and/or a Ligand Binding Assay. Other aliquots of the same cells are used to create paraffin- or agarose-embedded sections which are referred to as calibration samples. It is also possible, and, in some cases, desirable, to create a calibration sample that contains a mixture of distinct cell lines, so that two or more biomarkers of interest may be represented, or to replicate heterogeneity of biomarker expression, which is often typical of cancer tumors.

According to further aspects, having independently measured the amounts of analytes in each of the cell preparations by one or more standard techniques (flow cytometry or LBA, for example), other aliquots of the same cells are pelleted, paraffin- or agarose-embedded and sectioned or placed in a calibration array block for sectioning (to create the calibration array sections). Calibration arrays containing several different calibration samples (each representing a different cell line, expressing the desired biomarker at different levels, or expressing different biomarkers), are then placed on the slide. Since each calibration array is cut from the same block, each of the tissue section cores (from each cell pellet) contains the same distributions of analytes, thereby providing an inter-slide and inter-laboratory reference in addition to an intra-slide reference. While later production of a calibration array block from the same cell lines re-cultured and re-analyzed at a later time point may exhibit different amounts/distributions of analytes, those differences will all be measured with the validation method (e.g., flow cytometry). In addition, each batch of calibration arrays from a single calibration array block can be compared by combining them together on the same slide for measurements of the differences. Manufacturing these standardization calibration arrays in large numbers and supplying them to the clinical laboratories carrying out the diagnostic tests will prove cost effective and convenient.

Desirably, these calibration standards improve clinical practice by providing reliably consistent analytical/quantitative measurements of the amount of biomarker. Incorporation into every slide of a quantitative, independently measured linear calibration standard also simplifies confirmation that the processing did not destroy the linearity so much that it could not be corrected. This desirably advances the improvement and standardization of tissue preparation and processing procedures

In further aspects, the problem relating to the failure of constrained peptides to detect pre-analytic sources of error is solved by use of cells in model tissues subjected to all of the same procedures as the patient sample to be analyzed after fixation and paraffin embedding. Calibration array sections (used as controls) represent a check on antigen retrieval. When calibration array sections do not control for fixation errors, the use of a range of cell lines expressing different amounts of analytes represents the best possible standard.

In further aspects, the problem relating to the specificity of constrained peptides to a particular MAb is overcome by the use of calibration arrays, which reproduce the complete analyte in its native conformation inside a cell; these analogous characteristics enable the calibration array to respond virtually identically to the patient tissue during slide preparation and labeling.

Further, some aspects solve problems relating to the variability of cell pellet tissue microarrays (TMAs) and constrained peptides. (A TMA is an array of dozens to hundreds of tissue cores, mounted, in parallel, in the same block of paraffin). In this regard, the calibration array block can be placed directly in the same TMA block or tumor block as the patient biopsies. The calibration array block can then be sectioned along with the patient tissue to provide more uniform thicknesses to reduce this source of error. Different cell lines already exist that express different biomarker/analyte concentrations, but different cell lines can also exhibit different cell sizes; larger cells would be cut proportionally more, leaving less analyte per cell in the pellet section. When the size differences between cell lines are great enough to significantly alter the relative intensities as compared to the flow cytometry reference step, a different cell line with a more similar volume can be engineered to express the appropriate level of analyte or levels of multiple analytes. In short, the same parent cell line can be genetically engineered to produce many sub-lines, each with a different biomarker/analyte or combination of biomarkers/analytes. This enables appropriate control over the possible shift in relative intensities resulting from differences in the sizes of cell in each cell line.

On-slide calibration standards can include a calibration set constituted of several separate calibration samples (e.g., 3 to 12 or more separate samples). Each sample in a calibration set will correspond to a small circular, rectangular, or other shape, spot on the slide. The calibration samples are arrayed into a calibration array. Each spot contains cellular material that contains a desired biomarker that is present at a known amount within the spot. Certain spots are “blanks” and have none of the desired biomarker. Other spots have intermediate levels of the biomarker; still other spots have high levels of the biomarker. There is either a single spot corresponding to each level of biomarker, or there are multiple spots for each biomarker level (e.g., replicate samples, such as duplicates or triplicates, for each level of biomarker). Certain calibration standards feature expression of multiple biomarkers (e.g., 2, 3, or even more proteins of interest that serve as biomarkers for a particular tumor or disease).

Cell lines, mounted on slides, are used as calibration standards for quality control. Cell lines can be grown in vitro, harvested, formalin fixed, concentrated via centrifugation, and embedded in paraffin or agarose to prepare cell pellets. The cell pellets are then sectioned and mounted on glass microscope slides. The cell pellets, embedded in paraffin or agarose, will also be arrayed into an array of the pellets. In this regard, the pellets, which are cylindrical in shape, are mounted, together, in a single “block” sample (calibration array), which is analogous to a TMA. Cell lines offer the advantage of being available in potentially unlimited quantities, as they can typically be proliferated, indefinitely, and harvested in large batches. This feature overcomes a critical limitation of tissues as controls.

Different cell lines exhibit different levels of expression of desired biomarkers. An example is the use of MCF7 cells, which express high levels of ER, or SKBR3 cells that express high levels of HER2/neu. Certain cells can also serve as blanks for a particular biomarker. Furthermore, cell lines are included which express different levels of the same biomarker (for example, no expression, intermediate expression, or high expression), which enables provision of a calibration curve allowing protein expression in the biopsy or tissue section to be ascertained via comparison to the calibration array. Thus, different samples within the calibration array may contain material from different cell lines in order to provide a linear curve of expression of a single biomarker, or to provide calibration for multiple biomarkers.

The expression of the biomarker within the cell lines can be validated by other techniques (e.g., Western blotting, flow cytometry or ligand binding assays) in parallel with the preparation of the calibration standards for digital histochemistry. To do this, the cells are proliferated to the desired amount and a portion assayed for biomarker expression via the validation technique, with the rest of the preparation being used to produce the on-slide calibration samples. This serves as an additional quality control step to insure that the biomarker expression within the calibration samples is as expected. Cell preparations that do not express the biomarker at the desired level are not used to prepare the on-slide calibration samples.

Slides are prepared so that there is a large open area on the slide adjacent to the on-slide calibration samples. To use these slides for quantification of biomarkers in biopsy material or research tissue sections, the biopsy material or tissue section is placed on the slide, in the open area reserved for this purpose, which is near the calibration samples. Advantageously, this arrangement places the calibration samples near the test sample during the sample processing procedure; the calibration samples are therefore exposed to essentially the identical solutions and temperatures, and overall-environment, as the test sample. The placement of the calibration samples is convenient for reading of the samples by an automated microscopy system without switching slides between biopsy and calibration material to collect calibration data.

In another aspect, a calibration array is mounted directly in the same block as a patient biopsy, or a TMA block. The calibration array can then be sectioned along with the patient tissue or TMA so that the thickness of the calibration array will be the same thickness of the biopsy or TMA material, which may be desirable to correct for potential differences introduced by the cutting and mounting procedures.

These devices and procedures improve upon current clinical and research practice by providing reliably consistent analytical/quantitative measurements of the amount of biomarker. This particularly improves digital histocytometry procedures, as staining intensity for the biomarker, obtained from the sample, can be quantified by comparison to the calibration samples. The quantification of biomarkers via this method are useful, clinically, and, additionally for research and development purposes. For example, the calibration standards can be used to test the performance of new primary antibodies for a given biomarker, or to test different sample processing procedures, which may improve the overall procedure.

These devices and procedures also apply to quantifying the expression of biomarkers in material derived from other biological material, in addition to cell lines that are relevant to cancer. Thus, the possible test materials include human biopsy material or tissue sections, tissue sections from animal model systems used in biomedical research, or cellular material from any living organisms from the kingdoms of biology (Plantae, Fungi, Protista, Archaea, and Bacteria). Quantification of biomarker expression in non-human, non-animal organisms may have applications in agriculture, veterinary medicine, parasitology, microbiology, ecology, and environmental toxicology.

### BRIEF DESCRIPTION OF THE DRAWINGS

FIG. 1 is a schematic view of a process for assembling a calibration standard device for digital histochemistry.

FIG. 2 is a schematic view of how a calibration standard device with a test sample is processed.

FIG. 3 is a schematic that depicts how data obtained from a test sample can be quantitatively compared to data obtained from calibration samples that make up the calibration array.

FIGS. 4A, 4B, and 4C represent a more detailed analysis of biomarker expression that can be performed with the calibration standard device.

### DETAILED DESCRIPTION

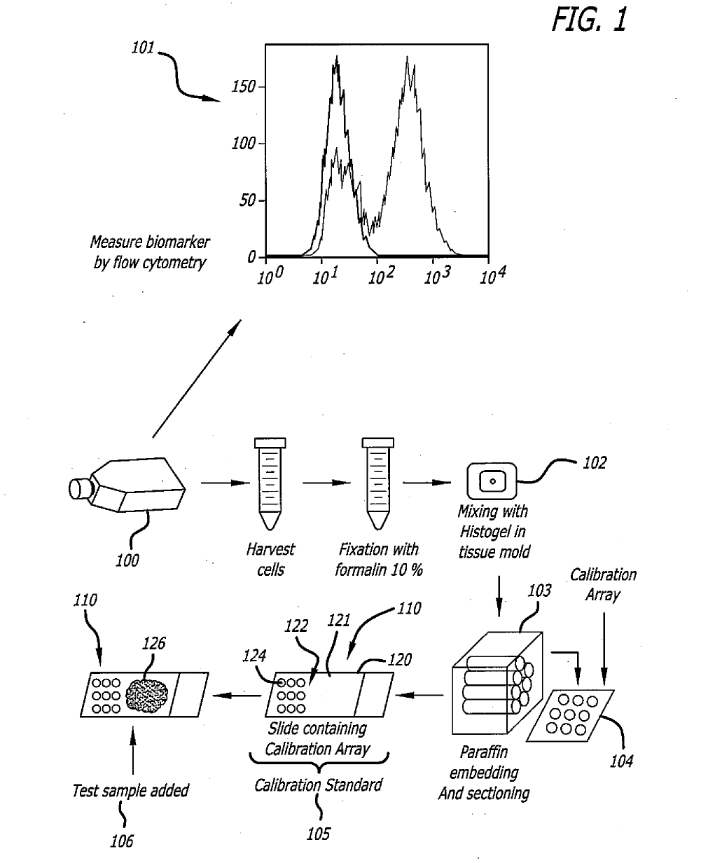

With reference to FIG. 1, cells which express desired biomarkers, such as those that are important to diagnose the types of breast tumors, are grown in flasks (100); the cells are then harvested and fixed. Concurrently, an aliquot of the preparation is analyzed by a validation method, such as flow cytometry (101) to confirm that the desired biomarker is expressed. Following fixation, the cells are mixed with histogel and molded to form cylindrical cell pellet, which makes up a calibration sample (102). The cell pellets are then embedded in a paraffin block (103), which can contain pellets representing several different cell lines (which express different biomarkers, or the same biomarker at different expression levels); the block is then sliced perpendicular to the axes of the cell pellets, to create a calibration array (104). A calibration standard device (105) is a microscope slide with the calibration array affixed to it, in such a manner that there is an empty space on the slide adjacent to the calibration array for receiving a test sample. The test sample (106) is added to the calibration standard device by the user.

Thus, at 105 in FIG. 1, a calibration standard device 110 is illustrated. The calibration standard device 110 includes a microscope slide 120 having a surface 121. A calibration sample 122 is disposed on a first portion of the surface 121 for quantification of biomarkers in a test sample. The calibration sample 122 includes a plurality of cell pellets 124 prepared from a living organism. At 106 of FIG. 1, a test sample 126 is added to the calibration standard device 110, and is disposed on a second portion of the surface 121, adjacent to the calibration sample 122. In some aspects, the test sample 126 includes one of biopsy material, a tissue section, a tissue microarray, and possibly, other materials. For example, a calibration sample 122 constituted of a TMA can be applied to the first portion of the surface 121 together with a test sample constituted of one or more sections from breast cancer tumors or TMAs containing arrays of breast cancer tumors obtained from tumor banks. In this regard, calibration TMAs can be used on assays from FFPET or fresh frozen tissue. An automated microscopy system equipped with tools of digital cytometry is used to perform selective analysis of regions of interest (ROIs) and automated analysis of TMAs with reference to a calibration TMA, Such a system is described in US 2010/0192084 A1, published on Jul. 29, 2010.

For illustrative purposes, an on-slide calibration standard allows the populations of cells expressing HER2/neu and ER, to be quantified in biopsies and tissue sections relevant to breast cancer. Breast cancer will thus provide a focused illustration of an on-slide calibration standard, which is general in that it works analogously on all tissues (cancers) and biomarkers. The preparation with the on-slide calibration standard starts with the culture of cell lines that express a biomarker of interest.

For example, relevant to breast cancer, MCF7 cells express ER, and SKBR3 cells express HER2/neu. Also of relevance, the cell line MDA-MB231 cells do not express either ER or HER2/neu, and can therefore function as a “blank” control.

Cells are cultured in flasks or related containers that are specially designed for this purpose. In culture, the cells multiply in a predictable manner. Once the desired number of cells is present, the cells are harvested from the flasks, by exposing the cells to an enzyme (trypsin), in a specialized solution that results in the cells detaching from the flask, and from each other. The cells are then fixed (exposed to formaldehyde, or related chemical compounds, that kill the cells and chemically cross link the cellular proteins and other material, in place).

At this point, a small aliquot of the cells is reserved for analysis of biomarker expression by a validation technique (e.g., flow cytometry, Western Blotting, or Ligand Binding Assay). If the cells do not express the biomarker at the appropriate level of expression, the preparation is discarded.

The remainder of the cell suspension is concentrated by centrifugation or related methods and resuspended in a solution that solidifies into a gel (histogel, agarose, or other gel material could be used). The preparation at this point is referred to as a cell pellet or “calibration sample”.

Calibration samples can be of any size; larger sizes are more expensive, so typical calibration samples are expected to be about 1 mm in diameter, even though we've prepared calibration samples larger in diameter (>4 mm). The calibration samples are generally cylindrical in shape. Multiple calibration samples representing either different cell lines (for example, representing MCF7 cells, SKBR3 cells, or MDA-MB231) or different preparations of the same cell type, can be affixed to each other so that the calibration samples are parallel to one another in a paraffin matrix. The block with the multiple calibration samples is then sliced to create a calibration array, which is mounted on a microscope slide. The calibration array is mounted on the microscope slide in such a manner that there is an open space adjacent to the calibration array. The open space will be where the sample (e.g., biopsy material, tissue sections, or other biological material) will be placed on the microscope slide. The microscope slide with the calibration array (105, FIG. 1) constitutes a calibration standard that can be provided to clinical laboratories and biomedical researchers to assist in quantification of biomarkers in human biopsy material, and in tissue sections from animal models of breast cancer.

Calibration arrays (104 in FIG. 1) can be constructed with different cell lines that express different biomarkers, to prepare calibration standards for different cancer types. More generally, calibration standards can be used to assist in quantifying the expression level of biomarkers in any biological sample, provided that cell lines are available that express the desired biomarkers or cell lines that express the desired biomarkers as a result of bioengineering.

To use a calibration standard, end-users will mount the test sample on the microscope slide adjacent to the calibration array (106 in FIG. 1). After this step, the slide, with the test sample plus the calibration standard, will be labeled for the desired biomarker (or multiple biomarkers). For example, the slide is incubate with a primary antibody, which binds to the biomarker within both the test sample and, also, to the biomarkers within the calibration samples of the calibration array. Following incubation with the primary antibody, the samples are further processed by incubation with a secondary antibody, which can be coupled to either an enzyme such as horse radish peroxidase, which enables labeling of the sample with a colorimetric marker, which can be viewed via bright field, or with a fluorescent chemical compound (e.g., fluorescein isothiocyanate), which can be viewed and quantified with fluorescence microscopy. Note that multiple biomarkers can be simultaneously labeled on the sample and calibration array via the use of appropriate primary antibodies and secondary reagents.

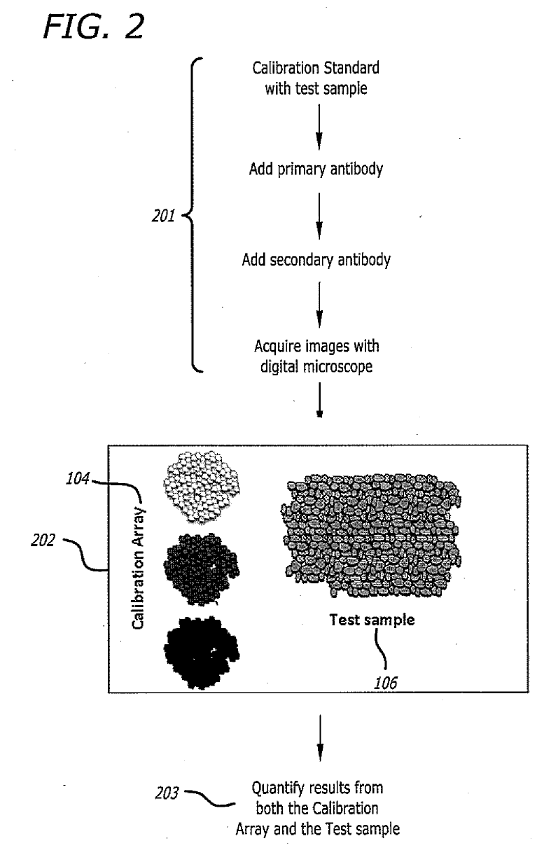

Following labeling of the test sample and calibration array for the biomarker(s), the slide is viewed and imaged under a microscope. With reference to FIG. 2, the desired biomarker (or multiple biomarkers) is labeled on the calibration standard using immunohistochemistry, or immunofluorescence reagents (201), and images (202) are acquired from the slide with a digital microscope. The different calibration samples that make up the calibration array (104) will label with different intensities for the biomarker; the test sample (106) will most likely label with an intensity that is within the range of intensities represented by the calibration samples. The images acquired from both the calibration array and the test sample are then analyzed (203) via image analysis software

It is envisioned that the most appropriate method for use of the system will be to photograph the test sample and calibration array, using a digital camera interfaced to the microscope. Following image acquisition, the images are analyzed for the intensity of the biomarker label, both in the test sample, and in the individual calibration samples of the calibration array. If the labeling is done for immunofluorescence, images from regions of the cells or tissues that do not express the biomarker will be dark, whereas regions where the biomarkers are expressed will be higher in intensity. If the test sample and calibration array is labeled for the intended use of immunohistochemistry for visualization in bright field, regions where the biomarker are absent will be light (transparent, due to lack of labeling), whereas regions where the biomarker is expressed will be dark (due to absorption of light from the deposited colored label).

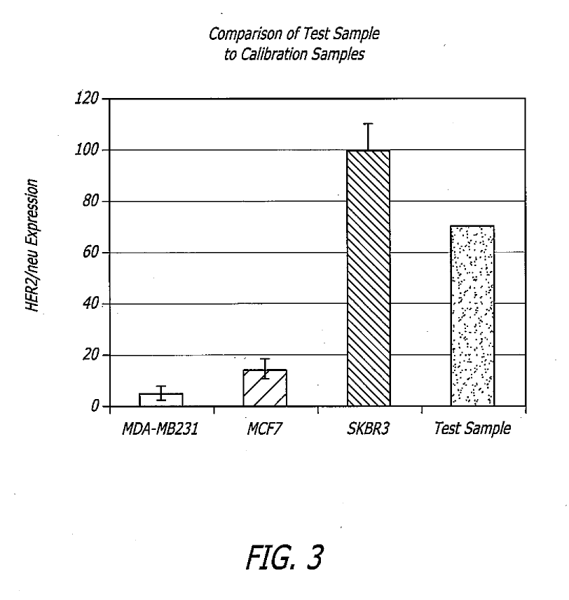

Following image acquisition, the digital images can be analyzed via image analysis software including general purpose image analysis programs such as Adobe Photoshop, or Image J (public domain, from the National Institutes of Health), or with programs designed specifically for cell image analysis (e.g., a CyteSeer® system from Vala Sciences, Inc). For images resulting from immunofluorescence, intensity values for each pixel in the images vary in accordance with the expression level of the labeled biomarker. The fluorescence intensity levels for cells within the test sample can therefore be compared to the intensity level for cells within the different calibration samples of the calibration array. In a typical implementation of this strategy, the average intensity of cells in the test sample for HER2/neu are compared to a calibration array derived from breast tumor cell lines that express HER2/neu at different levels (FIG. 3). In this manner, the HER2/neu expression of the test sample can be quantified vs. the HER2/neu expression in the reference cell lines, which affords a degree of quantification that is not attained with current practice. In the example illustrated in FIG. 3, the expression level of HER2/neu in the test sample is 70% of the HER2 expression found in the SKBR3 cell line, a cell line derived from a breast tumor.

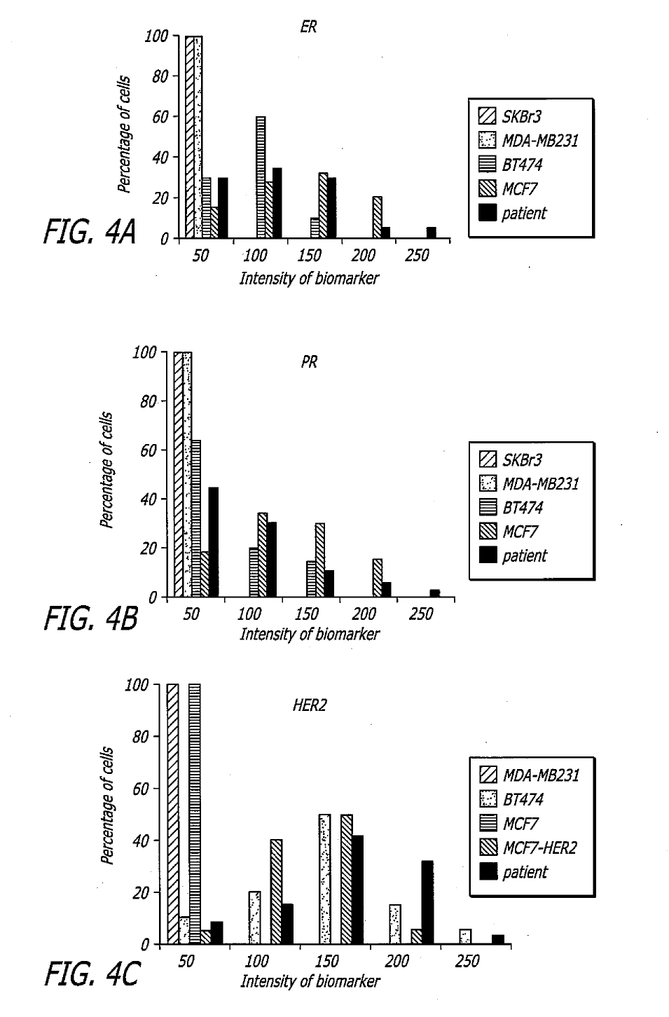

A second quantification scheme that can be performed with calibration standards is illustrated in FIGS. 4A, 4B, and 4C, which represent a more detailed analysis of biomarker expression that can be performed with the calibration standards. In this case, a breast tumor section from the same patient was labeled and compared to a calibration array constructed from breast tumor cell lines (SKBR3, MDA-MB231, BY474, and MCF7). The histograms depict the percentage of cells within the test sample vs. the calibration array, at 5 different intensity levels: 0 to 50, 51 to 100, 101 to 150, 151 to 200, and 201 to 250 intensity units/pixel, for ER (FIG. 4A), PR (FIG. 4B), and HER2/neu (FIG. 4C).

As seen, the sample from the patient was positive for ER and PR expression, and very positive for HER2/neu. These histograms graph the percentage of cells that express a given biomarker at different intensity levels for the test sample and the calibration array. For ER, the test sample is markedly positive for ER (as over 20% of the cells populate the 3^{rd} bin for ER) significantly positive, but less so, for PR (as just 7% of the cells populate the 3^{rd} bin); these results are similar to those obtained with the MCF7 cell line. For HER2/neu, the test sample is very positive (Over 30% of the cells populate the 4^{th} bin of the histogram, which exceeds percentage of cells in this bin for MCF7-HER2/neu cells, a cell line that has been bioengineered to over express HER2/neu). This strategy of analysis yields data that is similar to the traditional scoring system utilized by pathologists (e.g., in which patient biopsies are scored as “1”, “2”, “3”, or “4”) and has the further advantage of enabling comparison to reference cell lines prepared from breast tumor cells.

Constructions and uses of a calibration standard device have been described with reference to specific embodiments; nevertheless, various modifications can be made without departing from the essence of an invention illustrated by those embodiments. Accordingly, the invention is limited only by the following claims.

1. A calibration standard device, comprising:
a microscope slide having a surface;
a calibration sample disposed on a first portion of the surface for quantification of biomarkers in a test sample;
the calibration sample including a plurality of cell pellets constituted of cells that express at least one biomarker;
a test sample including one of biopsy material, a tissue section, and a tissue microarray disposed on a second portion of the surface, adjacent to the calibration sample.

2. The calibration standard device of claim 1, in which the cell pellets are analyzed to validate expression of the at least one biomarker.

3. The calibration standard device of claim 1, in which multiple cell pellets comprising either multiple samples of the same cell type, or samples representing different cell types, are arranged into an array.

4. The calibration standard device of claim 1, in which multiple cell pellets comprising either multiple samples of the same cell type, or samples representing different cell types, are arranged into an array.

5. A method of molecular measurement using the calibration standard device of any one of claims 1-4, comprising the automated-microscopy-system-executed steps of:
obtaining a data measurement of biological molecules in the calibration standard;
obtaining a data measurement of molecules in the test sample that are similar or identical to the biological molecules;
comparing the data measurement of biological molecules to the data measurement of the similar or identical molecules in the test sample; and
reporting results of the comparing step such that an amount of the molecules in the test sample is reported in relationship to an amount of biological molecules in the calibration standard.

6. The method of claim 5, providing a calibration curve using the data measurement of biological molecules in the calibration standard.

7. A method of making a calibration standard device, comprising:
growing cells which express at least one biomarker;
analyzing the cells to confirm expression of the at least one biomarker;
forming a calibration sample of cell pellets containing the cells;
embedding the cell pellets in a paraffin block;
creating a calibration array by slicing the paraffin block; and,
placing the calibration array on a microscope slide such that there is an empty area on the microscope slide adjacent to the calibration array for receiving a test sample.

8. The method of claim 7, further including placing a test sample in the empty area.

9. The method of claim 7, in which multiple cell pellets comprise either multiple samples of the same cell type, or samples representing different cell types.

10. The method of claim 7, further comprising arranging the cell pellets into an array.

11. A calibration standard device, comprising:
a plurality of cell pellets disposed on a surface of a microscope slide;
in which the cell pellets include at least two cell types with different levels of biomarker for each type of a plurality of biomarker types.

12. The calibration standard device of claim 11, in which the cell pellets are disposed in a tissue microarray applied to the surface of the microscope slide.

13. The calibration standard device of claim 11, in which an immunofluorescent stain applied to the cell pellets is used to indicate one or more of ER, PR and HER2 biomarkers on the same sample.
